(19) United States
(12) Patent Application Publication
(10) Pub. No.: US 2011/0281320 A1
(43) Pub. Date: Nov. 17, 2011

(54) DEVICE FOR ANALYZING NUCLEIC ACIDS AND APPARATUS FOR ANALYZING NUCLEIC ACIDS

(76) Inventors: Toshiro Saito, Ibraraki (JP); Kazumichi Imai, Ibaraki (JP)

(21) Appl. No.: 13/147,117

(22) PCT Filed: Jan. 18, 2010
(86) PCT No.: PCT/JP2010/000214

§ 371 Date: Jul. 29, 2011

(30) Foreign Application Priority Data
Jan. 30, 2009 (JP) 2009018936

(51) Int. Cl. C12N 11/14; C12M 1/34; C07H 21/00; C12M 1/40; C12N 11/00

(52) U.S. Cl. 435/176; 435/287.2; 435/174; 536/24.3

(57) ABSTRACT

An object of the present invention is to regularly align microparticles, on each of which a nucleic acid synthetase or a DNA probe capable of capturing a nucleic acid sample fragment is immobilized, on a support so as to improve throughput of nucleic acid analysis. The present invention relates to a method comprising immobilizing a nucleic acid synthetase, a DNA probe, or the like in advance to a microparticle, forming a pattern of metal pads each having a diameter smaller than the microparticle diameter with gold or the like on a support, and allowing a microparticle to be bound to the pads via a chemical bond. In addition, when the surfaces of microparticles are electrically charged, a pattern of metal pads each having a diameter equivalent to or larger than the microparticle diameter is formed with gold or the like on a support and a microparticle is allowed to be bound to the pads via a chemical bond. According to the present invention, many types of nucleic acid fragment samples can be regularly aligned at a high density and immobilized on a support. This allows high throughput analysis of nucleic acid samples. For example, if microparticles are immobilized at 1-micron pitches, a high density of 10⁶ nucleic acid fragments/emm² can be readily achieved.

### TECHNICAL FIELD

The present invention relates to a device for analyzing nucleic acids and an apparatus for analyzing nucleic acids.

### BACKGROUND ART

For nucleic acid analyzing devices, new techniques for sequencing DNA and RNA have been developed.

Today, with conventionally used methods based on electrophoresis, a DNA fragment or RNA sample for sequencing is subjected to reverse transcription reaction to synthesize a cDNA fragment sample, a dideoxy reaction is performed by the well-known Sanger method, electrophoresis is performed, and a molecular weight separation/development pattern is determined and analyzed.

In recent years, a method for determining sequence information about many fragments in parallel by immobilizing many DNA fragments as samples on a support has been suggested.

PCR performed on microparticles used as carriers carrying DNA fragments is disclosed in Nature 2005, Vol. 437, pp. 376-380. After PCR, microparticles carrying PCR-amplified DNA fragments are introduced into many holes having diameters adjusted to sizes of the microparticles which are formed on a plate, followed by pyrosequencing-based reading.

In addition, PCR performed on microparticles used as carriers carrying DNA fragments is disclosed in Genome Research 2008, Vol. 18, pp 1051-1063. After PCR, microparticles are distributed and immobilized on a glass support. An enzymatic reaction (ligation) is induced on the glass support. Sequence information about each fragment is obtained by incorporating a substrate containing a fluorescent dye into the fragment and detecting fluorescence.

Further, immobilization of many DNA probes having identical sequences on a smooth support is described in Science 2008, Vol. 320, pp. 106-109. After a DNA sample is cleaved, a DNA probe sequence and an adapter sequence complementary to the DNA probe sequence are added to one end of each DNA sample fragment. These are hybridized on a support and thus sample DNA fragments are individually immobilized at random on the support. In such case, a DNA elongation reaction is induced on the support such that a substrate containing a fluorescent dye is incorporated into each fragment, followed by washing of an unreacted substrate and fluorescence detection. Thus, sequence information about each sample DNA is obtained.

As described above, methods for determining sequence information about many nucleic acid fragments in parallel by immobilizing the many nucleic acid fragment samples on a smooth support have been developed. Such methods are being used in practice.

### SUMMARY OF THE INVENTION

### Problem to be Solved by the Invention

As a result of intensive studies conducted to improve the throughput of parallel analysis, the present inventors obtained the findings described below.

In order to further improve the throughput of parallel analysis as mentioned above, it is desirable for nucleic acid samples to be regularly aligned and immobilized on a smooth support at the maximum possible density. A method in which nucleic acid samples have been retained on microparticles is highly advantageous in terms of sample handling because the number of DNA fragments to be analyzed is vary large. A method in which microparticles each carrying a nucleic acid sample are distributed and immobilized on a smooth support can be easily carried out. However, it is significantly time-consuming to perform data processing to obtain numerical data from images of randomly distributed microparticles detected by a CCD camera upon sequencing by fluorescent detection.

According to a method in which a plate on which many holes have been formed is prepared and microparticles carrying nucleic acid samples are aligned on the plate, the number of DNA fragments that can be read per assay is determined depending on the diameters of holes formed on the plate, which is at most 10⁴ fragments/glass slide. In this case, throughput improvement is limited.

An object of the present invention is to regularly align microparticles, on each of which a nucleic acid synthetase or a DNA probe capable of capturing a nucleic acid sample fragment is immobilized, on a support so as to improve the throughput of nucleic acid analysis.

### Means for Solving Problem

The present invention relates to a method comprising immobilizing a nucleic acid synthetase, a DNA probe, or the like in advance to a microparticle, forming a metal pad pattern with gold or the like on a support, and allowing the microparticle to be bound to the pad via a chemical bond.

The term “chemical bond” used in the present invention refers to a bond such as a covalent bond, a coordination bond, an ion bond, or a hydrophobic bond.

### Effects of the Invention

According to the present invention, many types of nucleic acid fragment samples can be regularly aligned at a high density and immobilized on a support. This allows high throughput analysis of nucleic acid samples. For example, if microparticles are immobilized at 1-micron pitches, a high density of 10⁶ nucleic acid fragments/emm² can be readily realized.

### BRIEF DESCRIPTION OF THE DRAWINGS

FIG. 1 illustrates an example of the configuration of a device for analyzing nucleic acids.

FIG. 2 illustrates an example of a method for producing a device for analyzing nucleic acids.

FIG. 3 illustrates an example of a method for immobilizing probe molecules on microparticles using nucleic acids as the probe molecules for a device for analyzing nucleic acids.

FIG. 4 illustrates an example of an apparatus for analyzing nucleic acids comprising a device for analyzing nucleic acids.

### DETAILED DESCRIPTION OF THE INVENTION

In the Examples described below, disclosed is a device for analyzing nucleic acids comprising: microparticles each having a probe molecule capable of capturing a nucleic acid to be analyzed, and being regularly immobilized on a support; and adhesive pads at positions at which the microparticles are immobilized on the support, wherein the microparticles are bound to the adhesive pads via chemical bonds.

In addition, in the Examples, disclosed is an apparatus for analyzing nucleic acids to obtain nucleotide sequence information about the nucleic acid sample, comprising:

a device for analyzing nucleic acids comprising: microparticles each having a nucleic acid molecule capable of capturing a nucleic acid to be analyzed, and being regularly immobilized on a support; and adhesive pads at positions at which the microparticles are immobilized on the support, wherein the microparticles are bound to the adhesive pads via chemical bonds;

a means for supplying a nucleotide having a fluorescent dye and a nucleic acid sample to the device for analyzing nucleic acids;

a means for irradiating the device for analyzing nucleic acids with light; and

a means for detecting light emission of fluorescence from the fluorescent dye incorporated into a nucleic acid chain through a nucleic acid elongation reaction that is caused by the simultaneous presence of a nucleotide, a nucleic acid synthetase, and a nucleic acid sample on the device for analyzing nucleic acids.

Also, in the Examples, disclosed is a method for producing a device for analyzing nucleic acids, wherein the device comprises microparticles each having a probe molecule capable of capturing a nucleic acid to be analyzed, and being regularly immobilized on a support, comprising:

supplying the microparticles on which the probe molecule have been immobilized to a support comprising adhesive pads, thereby immobilizing the microparticles at the predetermined positions on the support.

In addition, in the Examples, it is disclosed that a single molecule of the probe molecules is immobilized on a single microparticle.

Further, in the Examples, it is disclosed that the probe molecule is a nucleic acid or a nucleic acid synthetase.

Furthermore, in the Examples, it is disclosed that the microparticles are made of a material selected from the group consisting of semiconductors and metals.

Moreover, in the Examples, it is disclosed that the adhesive pads are made of a material selected from the group consisting of gold, titanium, nickel, and aluminum.

Hereinafter, the above and other novel features and the effects of the present invention are described with reference to the drawings. Here, in order to help complete understanding of the present invention, specific embodiments of the present invention are described in detail. However, the present invention is not limited to the content described herein.

### EXAMPLES

### Example 1

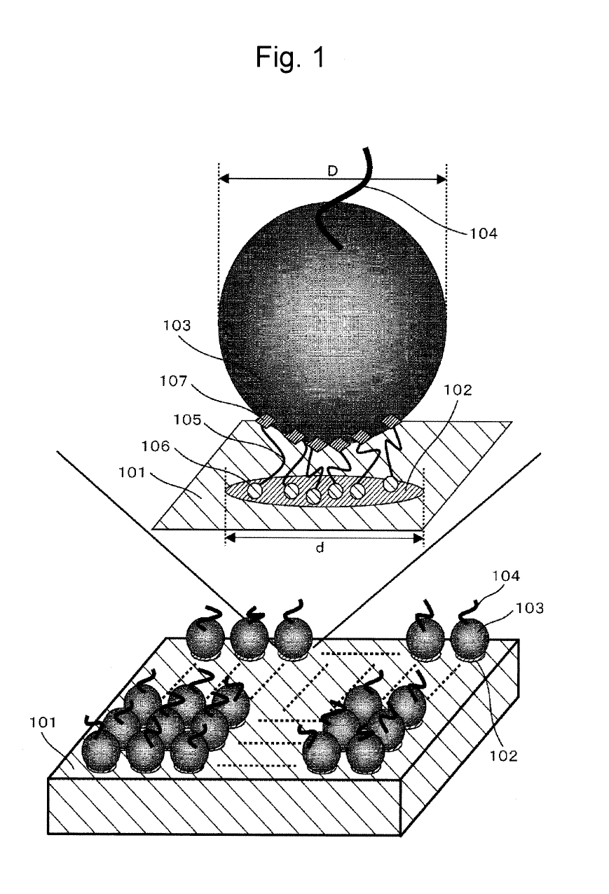

The configuration of the device used in this Example is described with reference to FIG. 1. Adhesive pads 102 may be regularly arranged in, for example, a grid form on a smooth support 101 as shown in FIG. 1. A microparticle 103 is chemically bound to an adhesive pad 102 via linear molecules 105. Preferably, a functional group 106 present at one end of each linear molecule 105 is bound to an adhesive pad 102 by chemical interaction. In such case, it is preferable that each functional group 106 weakly interacts with the smooth support 101 and strongly interacts with the adhesive pad 102. In view of the above, silica glass, sapphire, a silicon support, or the like can be used for the smooth support. In addition, the adhesive pad 102 may be made of a material selected from the group consisting of gold, titanium, nickel, and aluminum. The functional group 106 should be selected in consideration of a combination with the smooth support 101 and the adhesive pad 102. Examples of a functional group that can be used include a sulfhydryl group, an amino group, a carboxyl group, a phosphoric group, and an aldehyde group. The linear molecules 105 function to connect the microparticle 103 and the adhesive pad 102. The length of a linear molecule 105 is not particularly limited. However, when a low-molecular-weight molecule is used as a linear molecule 105, a linear molecule having approximately 3 to 20 carbon atoms is preferable. A functional group 107 present at one end of a linear molecule 105 causes adhesion between a linear molecule 105 and a microparticle 103. In addition, when a high-molecular-weight molecule is used as a linear molecule 105, a molecule having a plurality of side chains including side chains each having a functional group 106 and side chains each having a functional group 107 can be used.

Metal microparticles or semiconductor microparticles can be used as microparticles 103. For example, gold microparticles having diameters of 5 nm to 100 nm may be commercially available and thus can be used as appropriate. In addition, semiconductor microparticles of a compound semiconductor (e.g., CdSe) having diameters of approximately 10 nm to 20 nm may be commercially available and thus can be used as appropriate. Functional groups that can be used as functional groups 107 may differ depending on microparticle type. For instance, when gold microparticles are used, a sulfhydryl group, an amino group, or the like may be preferable. When semiconductor microparticles are used, commercially available microparticles with surfaces modified with streptavidin may be used. In such case, biotin can be used as a functional group 107. As a probe molecule 104 for capturing a nucleic acid, a single-stranded nucleic acid molecule such as DNA or RNA can be used. One end of the nucleic acid molecule is previously modified with a functional group 107 in the manner described above so that it is able to react with a microparticle 103. In addition, a nucleic acid synthetase can be used as a probe molecule 104 for capturing a nucleic acid. A reagent for introducing an avidin tag into an expressed protein is commercially available. For example, a nucleic acid synthetase can be readily immobilized on the surface of a semiconductor microparticle modified with, for example, a commercially available streptavidin by synthesizing a DNA polymerase with the use of such reagent. If a single-stranded nucleic acid molecule is used as a probe molecule 104 for capturing a nucleic acid, a sample nucleic acid molecule having a specific complementary sequence can be captured. After the capture of the nucleic acid, a nucleic acid elongation reaction can be induced on a support by supplying a nucleic acid synthetase and a nucleotide. If a nucleic acid synthetase is used as a probe molecule 104, a nonspecific sample nucleic acid molecule can be captured. Also in such case, a nucleic acid elongation reaction can be induced by supplying a nucleotide.

Preferably, a single molecule of probe molecule 104 is immobilized on a single microparticle 103. It is preferable for the particle diameter of a microparticle 103 to be minimized in order to immobilize a single molecule of probe molecule 104 on a single microparticle 103. This is because when a single molecule of nucleic-acid-capture probe molecule is immobilized on the surface of a microparticle 103, the electrically charged state of the microparticle surface varies, which causes inhibitory effects on an immobilization reaction of unimmobilized nucleic-acid-capture probe molecules onto the surface. Reduction of microparticle size promotes such inhibitory effects. As a result of intensive studies conducted by the present inventors, it has been found that microparticle size is preferably approximately 20 nm or less. In addition, a binding reaction between microparticles 103 and probe molecules 104 may be carried out in a liquid phase, and the concentration of a probe molecule 104 may be decreased to approximately 1/10 or less of that of a microparticle 103 for the reaction. Thus, even if the diameter of a microparticle 103 is approximately 1 μm, a single molecule of probe molecule 104 can be immobilized on a single microparticle 103. As the size of microparticle 103 increases, the density of probe molecules 104 immobilized on a support decreases. When probe identification is carried out via convenient fluorescence detection, the distance between probes may be preferably approximately 1 μm in view of the diffraction limit. Therefore, the appropriate size of a microparticle 103 may be 1 μm or less.

As a method for forming adhesive pads 102 on a smooth support 101, thin film processing, which has been practically used for semiconductors, can be employed. For instance, adhesive pads 102 can be prepared by vapor deposition/sputtering through a mask, or by vapor deposition/sputtering to form thin film, followed by dry or wet etching. Regular alignment of adhesive pads 102 can be readily achieved using thin film processing. The distance between pads can be appropriately adjusted. When fluorescent detection is performed using a detection means, the distance between pads may be preferably 500 nm or more in view of the diffraction limit of light detection.

After adhesive pads 102 have been formed on a smooth support 101, linear molecules 105 that connect microparticles 103 and adhesive pads 102 may be supplied to the adhesive pads so as to be immobilized thereon. For immobilization, in order to prevent nonspecific adsorption on the smooth support 101, it may be effective to carry out a method for reacting a material having strong adhesivity with the smooth support 101 with the smooth support 101 before supplying the linear molecules 105. For example, a silane coupling agent or the like can be used.

Next, a device for analyzing nucleic acids can be produced by supplying microparticles 103 on the surface of each of which a probe molecule 104 has been immobilized to the support and thereby immobilizing a microparticle 103 on each adhesive pad 102.

When the microparticles 103 are to be immobilized on the adhesive pads 102, more than one microparticle 103 could be immobilized on a single adhesive pad 102. If more than one microparticle 103 is immobilized thereon, information from different types of nucleic acid fragments are overlapping, making it impossible to conduct accurate nucleic acid analysis. Therefore, a single microparticle 103 should be immobilized on a single adhesive pad 102. The present inventors repeatedly conducted immobilization experiments under different conditions. As a result of intensive studies, the present inventors found that a single microparticle 103 can be immobilized on a single adhesive pad 102 if the diameter “d” of adhesive pad 102 is smaller than the diameter “D” of microparticle 103. Specifically, it can be explained that if a microparticle 103 having a size equivalent to or exceeding the size of an adhesive pad 102 is immobilized on the adhesive pad 102, the immobilized microparticle would cover unreacted linear molecules, which would prevent such molecules from reacting with other microparticles. In order to immobilize a single molecule of probe molecule 104 to a single microparticle 103, the diameter “D” of microparticle 103 may be preferably 20 nm or less, and thus the diameter “d” of adhesive pad 102 may be preferably 20 nm or less. As a result of further intensive studies, it has been found that when the surfaces of microparticles 103 are electrically charged, electrostatic repulsion may be present between the microparticles, and thus the number of microparticles immobilized on a single adhesive pad becomes 1 even if the diameter “d” of an adhesive pad 102 may be larger than the diameter “D” of a microparticle 103. Therefore, it has been elucidated that when the surface of a microparticle 103 is weakly electrically charged and thus electrostatic repulsion is weak, it is preferable for the diameter “d” of adhesive pad 102 to be smaller than the diameter “D” of microparticle 103. Also, when the surface of a microparticle 103 is strongly electrically charged and thus electrostatic repulsion is strong, it is not necessary for the diameter “d” of adhesive pad 102 to be smaller than the diameter “D” of microparticle 103.

There are various possible ways to detect information related to nucleic acid samples in the device for analyzing nucleic acids of this Example. In view of sensitivity and convenience, a method involving fluorescence detection may be preferably used. In such case, first, nucleic acid samples may be supplied to the device for analyzing nucleic acids so as to allow probe molecules 104 to capture the nucleic acid samples. Next, nucleotides each having a fluorescent dye are supplied thereto. If the probe molecules 104 are DNA probes, a nucleic acid synthetase may be supplied. A nucleic acid elongation reaction may be induced on the device, followed by fluorescent detection of the fluorescent dye incorporated into nucleic acid chains during the elongation reaction. In such case, a so-called sequential elongation reaction method can be readily achieved by supplying a single type of nucleotide and repeating the steps of washing unreacted nucleotides, observing fluorescent emissions, and supplying a different type of nucleotide. After observation of fluorescent emissions, fluorescence from the fluorescent dye may be quenched, or a nucleotide having a fluorescent dye at a phosphate moiety may be used to induce a continuous reaction. Thus, information on the nucleotide sequences of nucleic acid samples can be obtained. Alternatively, four types of nucleotides having different fluorescent dyes may be supplied and a continuous nucleic acid elongation reaction may be induced without washing, followed by continuous observation of fluorescent emissions. Thus, a so-called real-time reaction method can be realized. In this case, if a nucleotide having a fluorescent dye at a phosphate moiety may be used, the phosphate moiety may be cleaved after elongation reaction, and thus continuous fluorescent detections can be carried out without quenching to obtain information on the nucleotide sequences of nucleic acid samples.

Fluorescent emission can be enhanced for observation using, as the above microparticles, microparticles such as gold, silver, platinum, or aluminum microparticles having diameters of approximately 100 nm or less, on which localized plasmon excitation can be generated at a wavelength within the visible range. For example, fluorescence enhancement by surface plasmon of gold microparticles is reported in Nanotechnology, 2007, vol. 18, pp. 044017-044021. Fluorescence from a fluorescent dye bound to a nucleotide can be enhanced for fluorescent detection, and the signal/noise (S/N) level can be increased. Particularly when a nucleic acid synthetase is used as a probe molecule 104, a fluorescent dye can be continuously introduced into the electric field due to localized-plasmon, and stable fluorescence enhancement can be preferably achieved.

When semiconductor microparticles are used as the microparticles, semiconductor microparticles may be excited with light from an external light source. Then, the excitation energy may be transferred to a fluorescent dye bound to the incorporated nucleotide, allowing the observation of fluorescence from the fluorescent dye bound to each nucleotide. In this case, the excitation light source may excite only semiconductor microparticles. This is preferable because only a single type of light source is necessary.

When microparticles made of a polymeric material are used as the microparticles, the microparticle diameters can be uniformly adjusted. In addition, the microparticle diameters can be selected within a wide range from several tens of nanometers (nm) to several micrometers (μm). Further, the use of such microparticles may be preferable in that the amounts of functional groups introduced for an immobilization reaction of a probe molecule 104 onto a microparticle surface can be uniformly adjusted by modifying surface based on functional groups contained in the polymeric material. Particularly when a single molecule of probe molecule 104 is immobilized on a microparticle surface, the reproducibility of the immobilization rate may be very high and preferable.

### Example 2

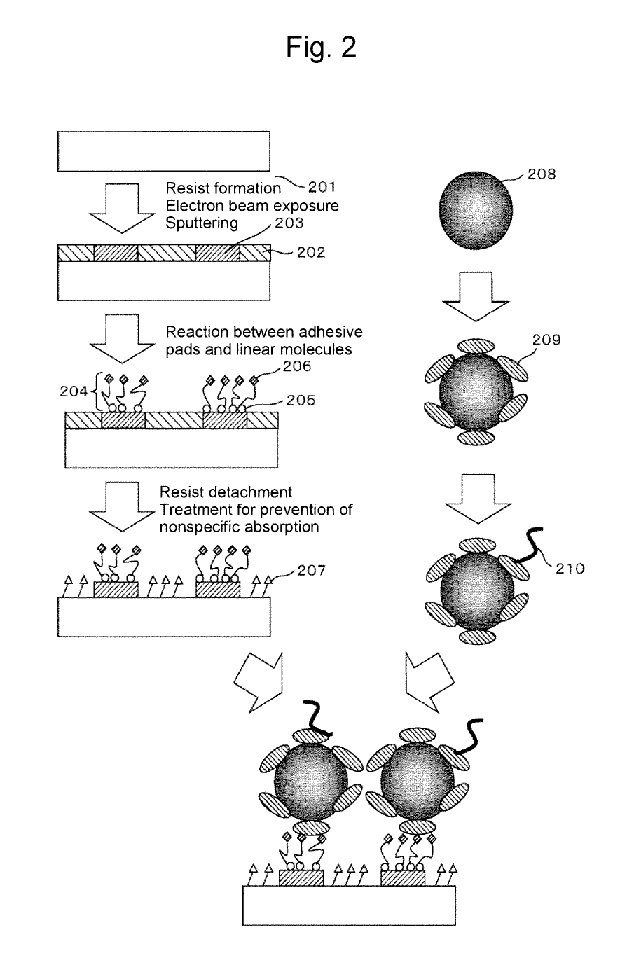

An example of a method for producing a device for analyzing nucleic acids is described below with reference to FIG. 2. A smooth support 201 may be coated with an electron beam positive-type resist 202 by spin coating. A glass support, sapphire support, silicon wafer, or the like can be used as a smooth support. If a smooth support incorporated into the device needs to be irradiated with excitation light from the back side opposite to the side upon which microparticles are aligned, a quartz support or a sapphire support having excellent light transmissibility may be used as a smooth support. Examples of an electron beam positive-type resist include polymethylmethacrylate and ZEP-520A (Zeon Corporation). Position adjustment can be carried out using the position of a marker on the support. Through holes with diameters of, for example, 15 nm may be formed on the resist by direct electron beam lithography. The pattern of the through holes may differ depending on the number of nucleic acid molecules that can be analyzed by parallel processing. It may be appropriate to form through holes with approximately 1-μm pitches in consideration of the ease of production, the yield improvement, and the number of nucleic acid molecules that can be analyzed by parallel processing. The through hole formation area may differ depending on the number of nucleic acid molecules that can be analyzed by parallel processing and also largely depending on the position accuracy and the position resolution of the detection means. For instance, in the case of arrangement of reaction sites (i.e., microparticles) with 1-μm pitches, 1,000,000 reaction sites can be formed within a through hole formation area of 1 mm×1 mm. After through hole formation, film formation may be carried out by sputtering using the material of the adhesive pads 203 (e.g., gold, titanium, nickel, or aluminum). When a glass support or a sapphire support is used as a smooth support, and gold, aluminum, or nickel is used as an adhesive pad material, it may be preferable to insert a titanium or chromium thin film between the support material and the adhesive pad material for enhancement of adhesion. Subsequently, a linear molecule 204 may be reacted with an adhesive pad 203. If the material for the adhesive pad 203 comprise gold, titanium, aluminum, or nickel, preferable examples of a functional group 205 present at one end of a linear molecule may include a sulfhydryl group, a phosphoric group, a phosphoric group, and a thiazole group, respectively. For example, biotin can be used as a functional group 206 present at the end opposite to the end at which a linear molecule is present. After linear molecules have reacted with the adhesive pad, the resist may be detached. After resist detachment, the surface of the smooth support (excluding each area in which an adhesive pad is formed) may be subjected to treatment for prevention of nonspecific adsorption. In order to prevent adsorption of a nucleotide having a fluorescent dye, the surface may be coated with molecules 207 for prevention of nonspecific adsorption, each having a negatively charged functional group. For example, the surface may be coated with epoxysilane by spin coating, followed by heat treatment and treatment with a weakly acidic solution (approximately pH 5 to pH 6). This causes ring-opening of epoxy groups and introduction of OH groups to the surface, and nonspecific adsorption prevention effects can be achieved.

Preferably, the surface of each microparticle 208 has been modified previously with avidin 209. When gold or platinum microparticles are used, modification with avidin can be readily carried out by reacting aminothiol, biotin-succinimide (NHS-Biotin; Pierce), and streptavidin with the microparticles in such order. If metal microparticles other than gold or platinum are used, the surfaces of the microparticles may be oxidized by heat treatment in an oxygen atmosphere. Thereafter, the metal microparticle surfaces can be readily modified with avidin by reacting aminosilane, biotin-succinimide (NHS-Biotin; Pierce), and streptavidin therewith in such order. If semiconductor microparticles are used as microparticles 208, commercially available microparticles can be used. For instance, microparticles having diameters of 15 to 20 nm (product name: ≡Qdot® streptavidin conjugate” (Invitrogen)) can be used. When an oligonucleotide is used as a nucleic-acid-capture probe 210, the oligonucleotide may be synthesized via terminal modification with biotin. Thus, such oligonucleotide can be readily immobilized on a microparticle. When a nucleic acid synthetase is used as a nucleic-acid-capture probe 210, an expression system may be first established using an RTS AviTag E. coli biotinylation kit (Roche Applied Science) to produce a nucleic acid synthetase. The thus produced nucleic acid synthetase can be readily immobilized on a microparticle.

A microparticle on which a nucleic-acid-capture probe is immobilized may be reacted with an adhesive pad. Thus, the device for analyzing nucleic acids of this Example can be produced.

### Example 3

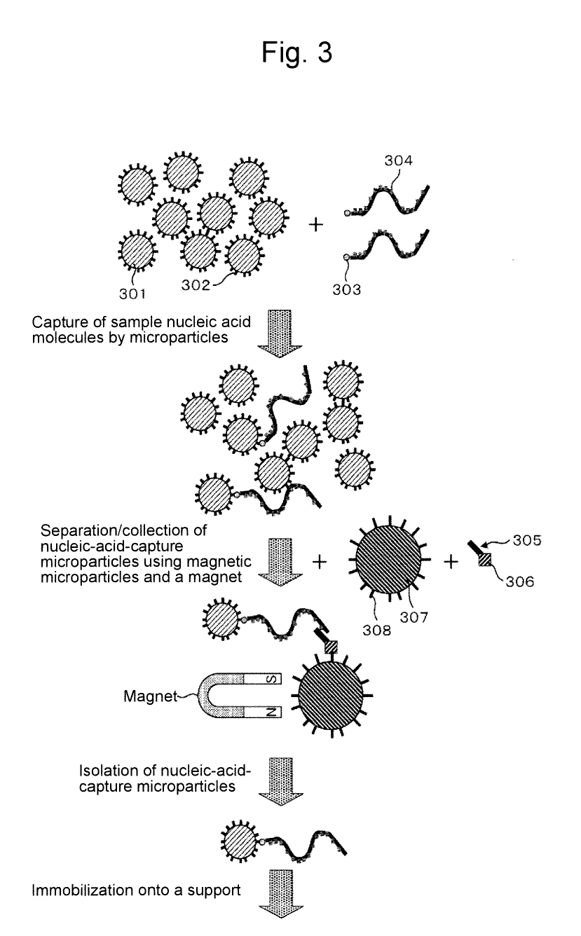

In this Example, an example of a method for producing a device for analyzing nucleic acids in which probe molecules are individually immobilized, and specifically, a method for immobilizing a single molecule of probe molecule on a single microparticle, is described with reference to FIG. 3. In this Example, a case in which a nucleic acid is used as a probe molecule is described. However, the method described herein can be similarly applied to a different probe molecule, such as a nucleic acid synthetase. A binding site 302 for capturing a sample nucleic acid molecule 304 may be previously bound to the surface of each microparticle 301. For example, streptavidin can be used as a binding site. Also, commercially available streptavidin-coated microparticles (Invitrogen) can be used as microparticles. A sample nucleic acid molecule 304 may be previously modified with binding sites 303. A binding site 303 can be selected from those can readily bind to a binding site 302 on the surface of a microparticle 301. For instance, when streptavidin is used as a binding site 302, biotin may be used as a binding site 303. One end of a sample nucleic acid molecule 304 can be easily ligated to a binding site 303 by synthesizing a PCR reaction product using a primer having terminal modification with a binding site 303 and a nucleic acid sample as a template. Next, a microparticle 301 may be reacted with a sample nucleic acid molecule 304 so as to allow the microparticle 301 to capture the sample nucleic acid molecule 304. In order to immobilize a single sample nucleic acid molecule 304 on a single microparticle 301, it may be preferable for the number of sample nucleic acid molecules 304 to be smaller than the number of microparticles 301 per unit of volume. This is because if the number of sample nucleic acid molecules 304 is excessively larger than the number of microparticles 301, it is highly probable that the number of sample nucleic acid molecules captured by a single microparticle 301 would be greater than 1. As a result of intensive studies conducted by the present inventors, it was found that when the number of microparticles 301 was 10 times the number of sample nucleic acid molecules 304 upon reaction, approximately 90% of microparticles 301 failed to capture sample nucleic acid molecules 304 while approximately 9% of microparticles 301 each captured a single sample nucleic acid molecule 304. The results are consistent with predictions based on the Poisson distribution assumption. Therefore, if microparticles 301 each capturing a sample nucleic acid molecule 304 may be exclusively collected, 90% or more of the collected microparticles 301 would have captured a single sample nucleic acid molecule 304. In order to collect such microparticles 301, a magnetic microparticle 307 is allowed to bind to each sample nucleic acid molecule 304 so as to collect the microparticles 301 using a magnet. In such case, an oligonucleotide 305 is prepared which has a sequence complementary to the terminal sequence of a sample nucleic acid molecule 304 and is terminally modified with a binding site 306 at one end. A magnetic microparticle 307 may be first subjected to surface coating to form a binding site 308 thereon such that binding takes place between a binding site 308 and a binding site 306. The sequence of an oligonucleotide 305 can be designed based on the primer sequence used for PCR amplification of a sample nucleic acid molecule 304. With the use of magnetic microparticles 307 prepared in the manner described above, microparticles 301 each capturing a single sample nucleic acid molecule 304 can be separated and collected at a high rate of 90% or more. In order to isolate nucleic-acid-capture microparticles 301 from magnetic microparticles 307, for example, denaturing treatment (high-temperature treatment) that causes separation between a double strand comprising a sample nucleic acid molecule 304 and an oligonucleotide 305 can be used. Isolated nucleic-acid-capture microparticles 301 can be immobilized at predetermined positions on a smooth support by the method described in Example 1. Thus, the device for analyzing nucleic acids of this Example in which sample nucleic acid molecules 304 are individually immobilized can be produced.

Further, in order to increase the proportion of microparticles each capturing a single sample nucleic acid molecule, it may be effective to employ electrophoresis. Briefly, based on the differences of the charge quantity on a microparticle depending on the number of nucleic acid molecules captured by the microparticle, microparticles each capturing nucleic acids may be allowed to migrate within gel (e.g., agarose gel) such that migration patterns can be obtained based on differences in the charge quantity corresponding to the number of captured nucleic acid molecules. Microparticles capturing no nucleic acids migrate the shortest distance. Microparticles each capturing a single nucleic acid molecule migrate the second-shortest distance. Thus, the corresponding band may be formed where the microparticles stop. Therefore, microparticles each capturing a single nucleic acid molecule can be obtained with high purity by excising the band.

### Example 4

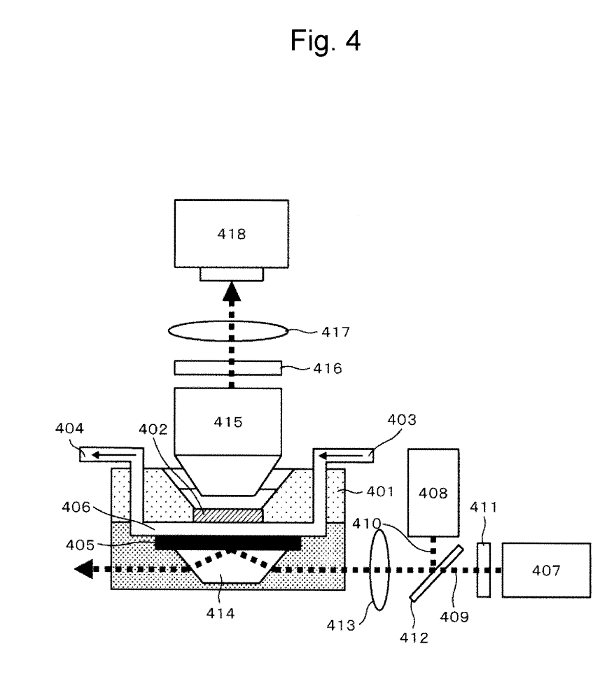

In this Example, a preferable configuration of an apparatus for analyzing nucleic acids comprising a device for analyzing nucleic acids is described with reference to FIG. 4.

The apparatus for analyzing nucleic acids of this Example comprises a means for supplying a nucleotide having a fluorescent dye, a nucleic acid synthetase, and a nucleic acid sample to a device for analyzing nucleic acids, a means for irradiating the device for analyzing nucleic acids with light, and a means for detecting light emission of fluorescence from the fluorescent dye incorporated into a nucleic acid chain through a nucleic acid elongation reaction that is caused by the simultaneous presence of a nucleotide, a nucleic acid synthetase, and a nucleic acid sample on the device for analyzing nucleic acids. More specifically, the device 405 may be installed in a reaction chamber comprising a cover plate 401, a detection window 402, an inlet 403, and an outlet 404, such inlet and outlet serving as solution-exchanging ports. PDMS (polydimethylsiloxane) may be used as a material for the cover plate 401 and the detection window 402. The thickness of the detection window 402 may be determined to be 0.17 mm. Laser light 409 and laser light 410 may be oscillated from a YAG laser light source 407 (wavelength: 532 nm; output: 20 mW) and a YAG laser light source 408 (wavelength: 355 nm; output: 20 mW), respectively. Laser light 409 alone may be circularly polarized using a λ/4 plate 411 so as to adjust the two laser light beams concentrically with a dichroic mirror 412 (for reflecting light with a wavelength of 410 nm or less), followed by light condensing with the use of a lens 413. Then, the device 405 may be irradiated with the light via a prism 414 with the relevant critical angle or greater.

An example in which gold microparticles each having a diameter of approximately 50 nm are used as microparticles is described below. In this case, localized surface plasmon may be generated on gold microparticles present on the surface of a device 405 via laser irradiation. Accordingly, a fluorophore of a target substance captured by a DNA probe bound to a gold microparticle is present in the enhanced electric field. A fluorophore may be excited with laser light, and the thus enhanced fluorescent emission may be partially output through the detection window 402. A parallel light beam may be formed with fluorescence passed through the detection window 402 using an objective lens 415 (×60; NA=1.35; operating distance: 0.15 mm). Then, background light and excitation light may be intercepted by an optical filter 416, resulting in imaging with a two-dimensional CCD camera 418 via an imaging lens 417.

An example of a nucleotide having a fluorescent dye that can be used in a sequential reaction system may be: a nucleotide in which a 3′-O-allyl group is added as a protective group at the 3′ OH position of ribose moiety and a fluorescent dye is bound via an allyl group at the 5-position of pyrimidine or the 7-position of purine as disclosed in P.N.A.S. 2006, vol. 103, pp. 19635-19640. The allyl group may be cleaved by light irradiation (e.g., wavelength: 355 nm) or by contact with palladium. Therefore, quenching of light emitted from a dye and control of an elongation reaction can be simultaneously achieved. Even in the case of a sequential reaction, there is no need to remove unreacted nucleotides by washing. In addition, since a washing step is not necessary, real-time measurement during an elongation reaction may also be achieved. In such case, there is no need to add a 3′-O-allyl group as a protective group at the 3′ OH position of ribose moiety in the above nucleotide. A nucleotide bound to a dye via a functional group that can be cleaved by light irradiation (at a wavelength of, for example, 355 nm) may be used.

Also, when semiconductor microparticles are used as microparticles herein, the above example of an apparatus for analyzing nucleic acids can be applied. For example, if a Qdot®565 conjugate (Invitrogen) is used as a semiconductor microparticle, sufficient excitation can be induced using a YAG laser light source 407 (wavelength: 532 nm; output: 20 mW). When the excitation energy is transferred to Alexa Fluor® 633 (Invitrogen) that cannot be excited with light at a wavelength of 532 nm, fluorescence emission takes place. Specifically, a dye bound to an unreacted nucleotide is not excited. Only after a nucleotide bound to a dye is captured by a DNA probe and thus becomes in proximity to a semiconductor microparticle, which results in energy transfer, light is emitted from the dye. Therefore, captured nucleotides can be identified by fluorescent detection.

As described above, when an apparatus for analyzing nucleic acids is constructed using the device for analyzing nucleic acids of this Example, analysis time can be shortened without introducing a washing step into the analysis process, and the device and the analysis apparatus can be simplified. Accordingly, not only sequential-reaction-system-based measurement but also real-time measurement can be achieved during a nucleotide elongation reaction. Thus, significant throughput improvement over conventional techniques can be realized.

### EXPLANATION OF REFERENCE NUMERALS

101: Smooth support
102: Adhesive pad
103, 208, 301: Microparticle
104: Probe molecule
105, 204: Linear molecule
106, 107, 205, 206: Functional group at one end of a linear molecule
201: Smooth support
202: Electron beam positive-type resist
203: Adhesive pad
207: Molecule for prevention of nonspecific adsorption
209: Avidin
210: Nucleic-acid-capture probe
302, 303, 306, 308: Binding site
304: Sample nucleic acid molecule
305: Oligonucleotide
307: Magnetic microparticle
401: Cover plate
402: Detection window
403: Inlet
404: Outlet
405: Device
406: Flow channel
407, 408: YAG laser light source
409, 410: Laser light
411: λ/4 plate
412: Dichroic mirror
413: Lens
414: Prism
415: Objective lens
416: Optical filter
417: Imaging lens
418: Two-dimensional CCD camera CLAIMS

1. A device for analyzing nucleic acids comprising:
microparticles each having a probe molecule capable of capturing a nucleic acid to be analyzed, and being regularly immobilized on a support; and
adhesive pads at positions at which the microparticles are immobilized on the support,
wherein the microparticles are bound to the adhesive pads via chemical bonds.

2. A device for analyzing nucleic acids comprising:
microparticles each having a probe molecule capable of capturing a nucleic acid to be analyzed, and being regularly immobilized on a support; and
adhesive pads at positions at which the microparticles are immobilized on the support,
wherein the microparticles are bound to the adhesive pads via chemical bonds, and
wherein the diameters of the adhesive pads are equivalent to or smaller than the diameters of the microparticles.

3. A device for analyzing nucleic acids comprising:
microparticles each having a probe molecule capable of capturing a nucleic acid to be analyzed, and being regularly immobilized on a support, the surfaces of the microparticles being electrically charged; and
adhesive pads at positions at which the microparticles are immobilized on the support,
wherein the microparticles are bound to the adhesive pads via chemical bonds, and
wherein the diameters of the adhesive pads are equivalent to or larger than the diameters of the microparticles.

4. The device for analyzing nucleic acids according to claim 1, 2, or 3, wherein a single molecule of the probe molecules is immobilized on a single microparticle.

5. The device for analyzing nucleic acids according to claim 1, 2, or 3, wherein the probe molecules comprise a nucleic acid or a nucleic acid synthetase.

6. The device for analyzing nucleic acids according to claim 1, 2, or 3, wherein the microparticles comprise a material selected from the group consisting of semiconductors and metals.

7. The device for analyzing nucleic acids according to claim 1, 2, or 3, wherein the microparticles comprise a polymeric material.

8. The device for analyzing nucleic acids according to claim 1, 2, or 3, wherein the adhesive pads comprise a material selected from the group consisting of gold, titanium, nickel, and aluminum.

9. An apparatus for analyzing nucleic acids to obtain nucleotide sequence information about the nucleic acid sample, comprising:
a device for analyzing nucleic acids comprising: microparticles each having a nucleic acid molecule capable of capturing a nucleic acid to be analyzed, and being regularly immobilized on a support; and adhesive pads at positions at which the microparticles are immobilized on the support, wherein the microparticles are bound to the adhesive pad via chemical bonds;
a means for supplying a nucleotide having a fluorescent dye and a nucleic acid sample to the device for analyzing nucleic acids;
a means for irradiating the device for analyzing nucleic acids with light; and
a means for detecting light emission of fluorescence from the fluorescent dye incorporated into a nucleic acid chain through a nucleic acid elongation reaction that is caused by the simultaneous presence of a nucleotide, a nucleic acid synthetase, and a nucleic acid sample on the device for analyzing nucleic acids.

10. An apparatus for analyzing nucleic acids to obtain nucleotide sequence information about the nucleic acid sample, comprising:
a device for analyzing nucleic acids comprising: microparticles each having a nucleic acid molecule capable of capturing a nucleic acid to be analyzed, and being regularly immobilized on a support; and adhesive pads at positions at which the microparticles are immobilized on the support, wherein the microparticles are bound to the adhesive pad via chemical bonds, and wherein the diameters of the adhesive pads are equivalent to or smaller than the diameters of the microparticles;
a means for supplying a nucleotide having a fluorescent dye and a nucleic acid sample to the device for analyzing nucleic acids;
a means for irradiating the device for analyzing nucleic acids with light; and
a means for detecting light emission of fluorescence from the fluorescent dye incorporated into a nucleic acid chain through a nucleic acid elongation reaction that is caused by the simultaneous presence of a nucleotide, a nucleic acid synthetase, and a nucleic acid sample on the device for analyzing nucleic acids.

11. An apparatus for analyzing nucleic acids to obtain nucleotide sequence information about the nucleic acid sample, comprising:
a device for analyzing nucleic acids comprising: microparticles each having a nucleic acid molecule capable of capturing a nucleic acid to be analyzed, and being regularly immobilized on a support, the surfaces of the microparticles being electrically charged; and adhesive pads at positions at which the microparticles are immobilized on the support, wherein the microparticles are bound to the adhesive pad via chemical bonds, and wherein the diameters of the adhesive pads are equivalent to or larger than the diameters of the microparticles;
a means for supplying a nucleotide having a fluorescent dye and a nucleic acid sample to the device for analyzing nucleic acids;
a means for irradiating the device for analyzing nucleic acids with light; and
a means for detecting light emission of fluorescence from the fluorescent dye incorporated into a nucleic acid chain through a nucleic acid elongation reaction that is caused by the simultaneous presence of a nucleotide, a nucleic acid synthetase, and a nucleic acid sample on the device for analyzing nucleic acids.

12. The apparatus for analyzing nucleic acids according to claim 9, 10, or 11, wherein a single molecule of the probe molecules is immobilized on a single microparticle.

13. The apparatus for analyzing nucleic acids according to claim 9, 10, or 11, wherein the probe molecules comprise a nucleic acid or a nucleic acid synthetase.

14. The apparatus for analyzing nucleic acids according to claim 9, 10, or 11, wherein the microparticles comprise a material selected from the group consisting of semiconductors and metals.

15. The apparatus for analyzing nucleic acids according to claim 9, 10, or 11, wherein the microparticles comprise a polymeric material.

16. The apparatus for analyzing nucleic acids according to claim 9, 10, or 11, wherein the adhesive pads comprise a material selected from the group consisting of gold, titanium, nickel, and aluminum.

17. A method for producing a device for analyzing nucleic acids, wherein the device comprises microparticles each having a probe molecule capable of capturing a nucleic acid to be analyzed, and being regularly immobilized on a support, comprising:
supplying the microparticles on which the probe molecules have been immobilized to a support comprising adhesive pads, thereby immobilizing the microparticles at the predetermined positions on the support.

18. The method for producing a device for analyzing nucleic acids according to claim 17, wherein a single molecule of the probe molecules is immobilized on a single microparticle.
